(19) United States
(12) Patent Application Publication
(10) Pub. No.: US 2002/0114854 A1
(43) Pub. Date: Aug. 22, 2002

(54) Chinese herb medicine composition for cancer prophylaxis and therapy

(76) Inventor: Ku Jeng, Taipei (TW)

Correspondence Address: VOLPE AND KOENIG, P.C., SUITE 400, ONE PENN CENTER, 1617 JOHN F. KENNEDY BOULEVARD, PHILADELPHIA, PA, 19103, US

(21) Appl. No.: 10/078,740

(22) Filed: Feb. 19, 2002

(30) Foreign Application Priority Data
Feb. 21, 2001 (TW) 090103977

(51) Int. Cl. A61K 35/78

(52) U.S. Cl. 424/764; 424/778

(57) ABSTRACT

A Chinese herb medicine composition for a cancer prophylaxis and therapy includes Honeysuckle Flower, Mother Chrysanthemum Flower, Dandelion Herb, Viola Yedoensis and Semiaquilegia adosoides (DC) makino. The Chinese herb medicine composition is composed with a special ratio, and the special ratio of Honeysuckle Flower: Mother Chrysanthemum Flower: Dandelion Herb: Viola Yedoensis: Semiaquilegia adosoides (DC.) makino by weight is 1:1:1:1:1.

### FIELD OF THE INVENTION

The present invention relates to a Chinese herb medicine composition for a cancer prophylaxis and therapy, and more particularly to a Chinese herb medicine composition for the prophylaxis and the therapy of cervical cancer, esophagus cancer, ovary cancer or bladder cancer.

### BACKGROUND OF THE INVENTION

For the cancer therapy in the current medical technology, the radiotherapy and the chemotherapy are widely used as well as the surgical operation. However, the radiotherapy or the chemotherapy will cause many ill side-effects such as nauseating, hair-losing, immunity-declining, during the period of treatment.

In addition, the anti-cancer drugs also play an important role in the cancer therapy. Similarly, the current anti-cancer drugs, such as Bleomycin and cis-platine widely used in clinical therapy, will result in many ill side-effects such as nauseating and allergic symptom.

A particular formula of Chinese medicine cited from a traditional Chinese pharmacopoeia is consisted of five Chinese herbs including Honeysuckle Flower, Mother Chrysanthemum Flower, Dandelion Herb, Viola Yedoensis and Semiaquilegia adosoides (DC.) makino. The particular formula has been used to cure many malignant furuncles and local inflamed, fever and pain for a long time. Currently, this particular formula is widely applied in the therapies of cellular tissue inflammation, suppurative tonsillitis, lobar pneumonia, acute renal pelvis nephritis, endocarditis, marrow inflammation and mastitis.

For the aspect of cancer therapy, even though the traditional Chinese herb is thought to be a potential anti-cancer drug, there are no substantial evidences to prove the effectiveness on the cancer.

Therefore, the purpose of the present invention is to develop a Chinese herb medicine composition to deal with the above situations encountered in the prior art.

### SUMMARY OF THE INVENTION

It is therefore an object of the present invention to propose a Chinese herb medicine composition applied in the cancer prophylaxis and therapy in accordance with the inhibition effect on the cancer cell.

It is therefore another object of the present invention to propose a Chinese herb medicine composition applied in the cancer prophylaxis and therapy without ill side-effects resulted from the clinical anti-cancer drugs.

It is therefore an additional object of the present invention to propose a Chinese herb medicine composition applied in the cancer prophylaxis and therapy with safety.

It is therefore an additional object of the present invention to propose a Chinese herb medicine composition applied in the cancer prophylaxis and therapy and having convenience in using and storing.

According to the present invention, there is proposed a Chinese herb medicine composition for a cancer prophylaxis and therapy including Honeysuckle Flower, Mother Chrysanthemum Flower, Dandelion Herb, Viola Yedoensis and Semiaquilegia adosoides (DC.) makino.

Preferably, the Chinese herb medicine composition is composed with a special ratio, and the special ratio of Honeysuckle Flower:Mother Chrysanthemum Flower:Dandelion Herb:Viola Yedoensis:Semiaquilegia adosoides (DC.) makino by weight is 1:1:1:1:1.

The cancer includes cervical cancer, esophagus cancer, ovary cancer or bladder cancer.

Certainly, the Chinese herb medicine composition can be an extracted liquid or a dry powder. The dry powder is preferably formed from the extracted liquid by freeze drying.

Certainly, the extracted liquid can be a direct extracted liquid or an extracted concentrate. Certainly, the extracted liquid can be used directly or after diluting.

Preferably, the Chinese herb medicine composition is used by orally taking, external using or the combination.

According to the present invention, there is proposed a Chinese herb medicine composition for a cancer prophylaxis and therapy including Dandelion Herb.

Preferably, the composition further includes Honeysuckle Flower, Mother Chrysanthemum Flower, Viola Yedoensis and Semiaquilegia adosoides (DC.) makino. The Chinese herb medicine composition is preferably composed with a special ratio, and the special ratio of Honeysuckle Flower:Mother Chrysanthemum Flower:Dandelion Herb:Viola Yedoensis:Semiaquilegia adosoides (DC.) makino by weight is 1:1:1:1:1.

The present invention may best be understood through the following description with reference to the accompanying drawings, in which:

### BRIEF DESCRIPTION OF THE DRAWINGS

FIG. 1A is a bar chart illustrating the survival rate of an esophagus cancer cell strain, CE-81, treated by cis-Platine for 24-96 hours according to the present invention;

FIG. 1B is a bar chart illustrating the survival rate of an esophagus cancer cell strain, CE-81, treated by a Chinese herb medicine composition for 24-96 hours according to the present invention;

FIG. 2A is a bar chart illustrating the survival rate of a cervical cancer cell strain, CC-135, treated by cis-Platine for 24-96 hours according to the present invention;

FIG. 2B is a bar chart illustrating the survival rate of a cervical cancer cell strain, CC-135, treated by a Chinese herb medicine composition for 24-96 hours according to the present invention;

FIG. 3A is a bar chart illustrating the survival rate of an ovary cancer cell strain, OC-117, treated by cis-Platine for 24-96 hours according to the present invention;

FIG. 3B is a bar chart illustrating the survival rate of an ovary cancer cell strain, OC-117, treated by a Chinese herb medicine composition for 24-96 hours according to the present invention;

FIG. 4A is a bar chart illustrating the survival rate of a bladder cancer cell strain, Bladder-3, treated by cis-Platine for 24-96 hours according to the present invention;

FIG. 4B is a bar chart illustrating the survival rate of a bladder cancer cell strain, Bladder-3, treated by a Chinese herb medicine composition for 24-96 hours according to the present invention;

FIG. 5A is a bar chart illustrating the survival rate of a cervical cancer cell strain, CC-7T, treated for 6 hours by an extracted liquid of Chinese herb medicine composition stored for different days according to the present invention; and

FIG. 5B is a bar chart illustrating the survival rate of a cervical cancer cell strain, CC-7T, treated for 8 hours by an extracted liquid of Chinese herb medicine composition stored for different days according to the present invention.

### DETAILED DESCRIPTION OF THE PREFERRED EMBODIMENT

Regarding to the cell strain culture in following embodiments, the culture solution of Ham's F 12/DMEM (v/v 1:1) including 10% (v/v) fetus cattle serum (FCS) and antibiotics (100 μg/ml streptomycin, 100 IU/ml penicillin, 1 μg/ml amphotericin) is used for cultivating all cell strains at 37° C., 5% CO₂. When the cell grows to 80% full, the cell is washed down by Trypsin-EDTA to do the sub-culture. For the sub-culture, the cell is rinsed by HBSS first, washed down by Trypsin-EDTA, and centrifugated at 1000 rpm, 25° C. for 10 minutes. Sequentially, the cell is diluted by four times culture solution to be continuously cultivated. In addition, the sub-culture is done once per week.

Referring to the preparation of the Chinese herb medicine composition, 20 g Chinese herb medicine composition including 4 g of Honeysuckle Flower, 4 g of Mother Chrysanthemum Flower, 4 g of Dandelion Herb, 4 g of Viola Yedoensis and 4 g of Semiaquilegia adosoides (DC.) makino, and 200 ml distilled water are heated at 100° C. in a water bath for 2 hours, and then cooled to 37-40° C. and filtered by suction. Sequentially, the filtrate is concentrated to half volume and autoclaved at 121° C., 1.2 atm, for 20 minutes. Finally, the concentrate is continuously diluted to 1/10,1/100 and 1/1000 by 0.9% NaCl or fetus cattle serum-free (FCS-free) culture solution for following experiments. In addition, the anti-cancer drugs, i.e. cis-Platine and Bleomycin, is used to be a control test of the embodiment.

### Embodiment 1

The esophagus cancer cell strain, CE-81, is washed down by Trypsin-EDTA, centrifugated, and diluted to a cell concentration of 106 cells/ml by FCS-free culture solution. After above treatments, the cell strain CE-81 is inoculated into a 96-well culture plate. Each well containing 100 ml cell strain CE-81 is cultivated at 37° C., 5% CO₂ for 48 hours. Sequentially, a diluted cis-Platine and a diluted Chinese herb medicine composition extract liquid from the previous preparation are added into the wells respectively to reach the final medicine concentration to 1/10,1/100 and 1/1000 of the Chinese herb medicine composition extract preparation. Then, the 96-well culture plate having the cell strain CE-81 treated by cis-Platine or the Chinese herb medicine composition extract liquid with different concentrations is cultivated at 37° C., 5% CO₂ for 24, 48, 72 and 96 hours, respectively. Each treatment is triplicated. After different treating time, the cell strain CE-81 is added 20 ml cell activity detecting agent CTAQTM, and the cell activity is detected at OD 492 nm by multiple frequency scanner. In addition, the standard curve of the cell strain CE-81 is constructed by using 100%, 80%, 60%, 50%, 40%, 20% t and 0% cell number to be cultivated at the same time. Thus, the survival rate of cell (%) is obtained via comparing the determined value difference between after and before adding CTAQ with the standard curve by intercalation.

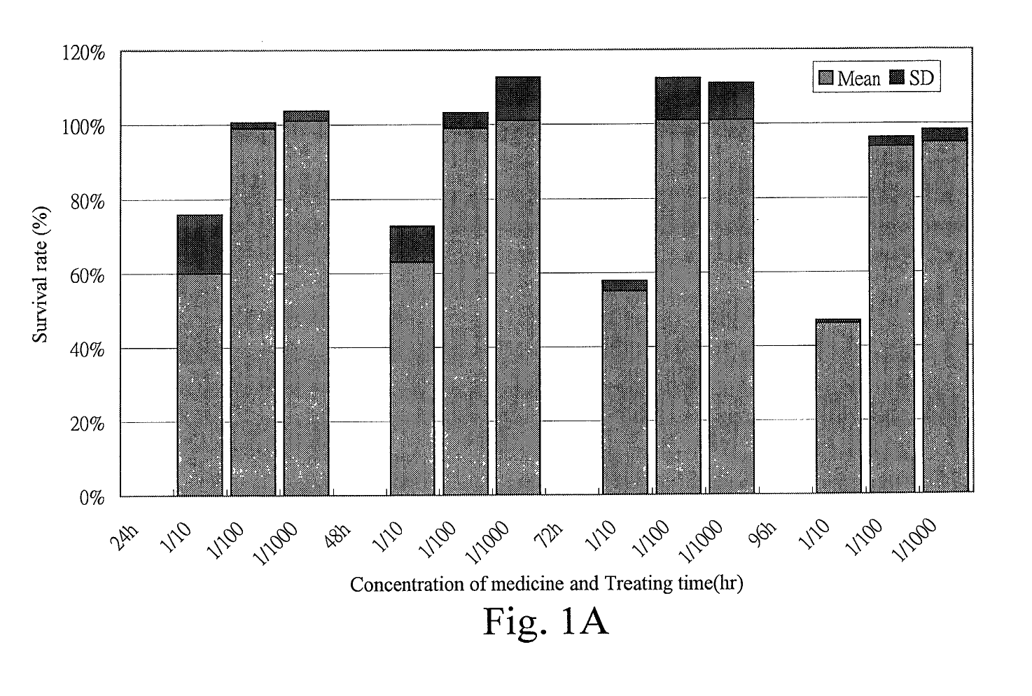

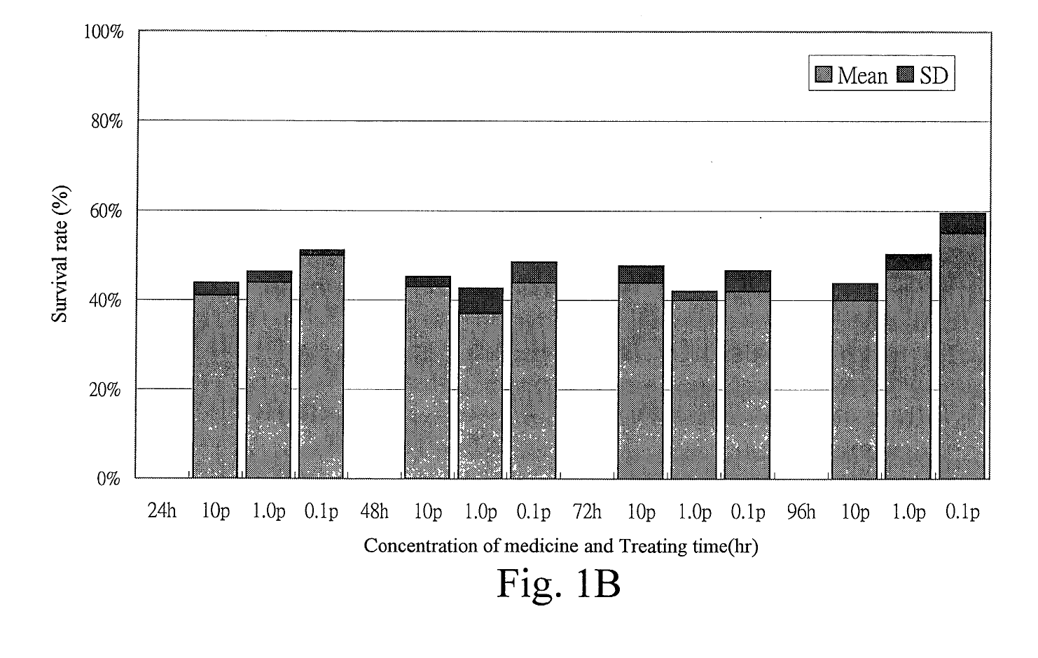

FIGS. 1A and 1B are bar charts illustrating the survival rate of an esophagus cancer cell strain, CE-81, treated by cis-Platine or the Chinese herb medicine composition for 24-96 hours according to the present invention. The final concentrations of the Chinese herb medicine composition are 1/10,1/100 and 1/1000 of the extract, respectively. For cis-Platine, the concentrations are 10 times (10 p), 1.0 times (1.0 p) and 0.1 times (0.1 p) of the highest concentration in blood, respectively, i.e. 20, 2.0 and 0.2 mg/ml. As shown in FIG. 1A, when the esophagus cancer cell strain CE-81 is treated by 10 p anti-cancer drug cis-Platine for 96 hours, the survival rate of CE-81 is still about 60%. On the contrary, when the esophagus cancer cell strain CE-81 is treated by 1/10Chinese herb medicine composition extract for 48-72 hours, the survival rate of CE-81 is also about 60%. However, when the treating time increased to 96 hours, the inhibition effect of 1/10 Chinese herb medicine composition extract for CE-81 is about 50% as shown in FIG. 1B. Therefore, the Chinese herb medicine composition has significant inhibition effect on the esophagus cancer cell.

Embodiment 2

In this embodiment, the methods for cell culture, sub-culture, experimental treatments, cell activity determination and calculation of the cell survival rate are the same as those of the Embodiment 1 except the cell strain is a cervical cancer cell strain CC-135.

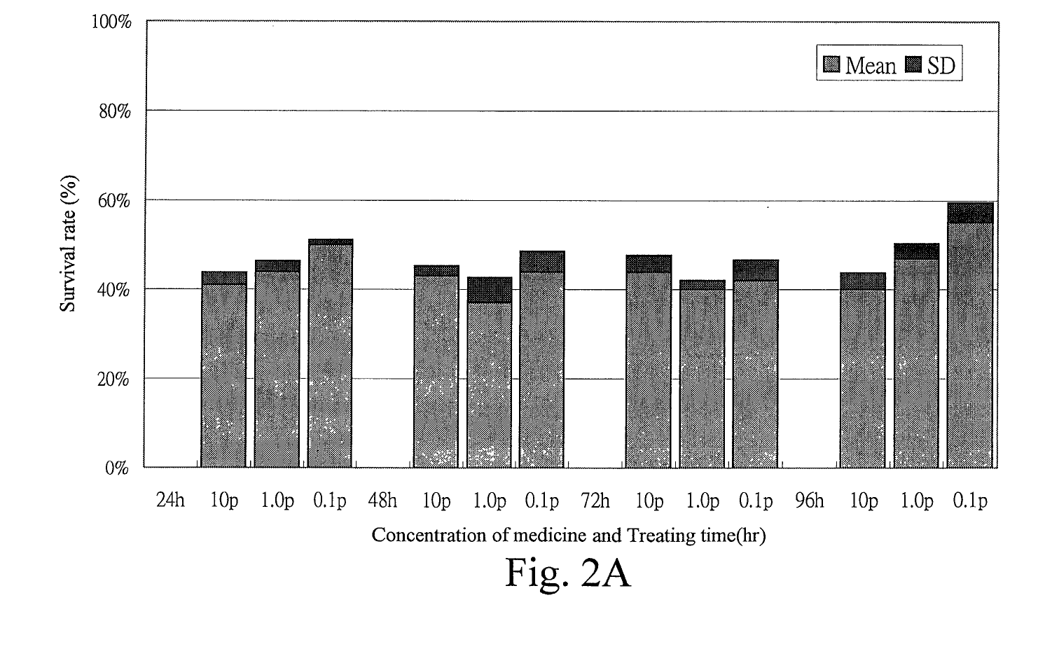

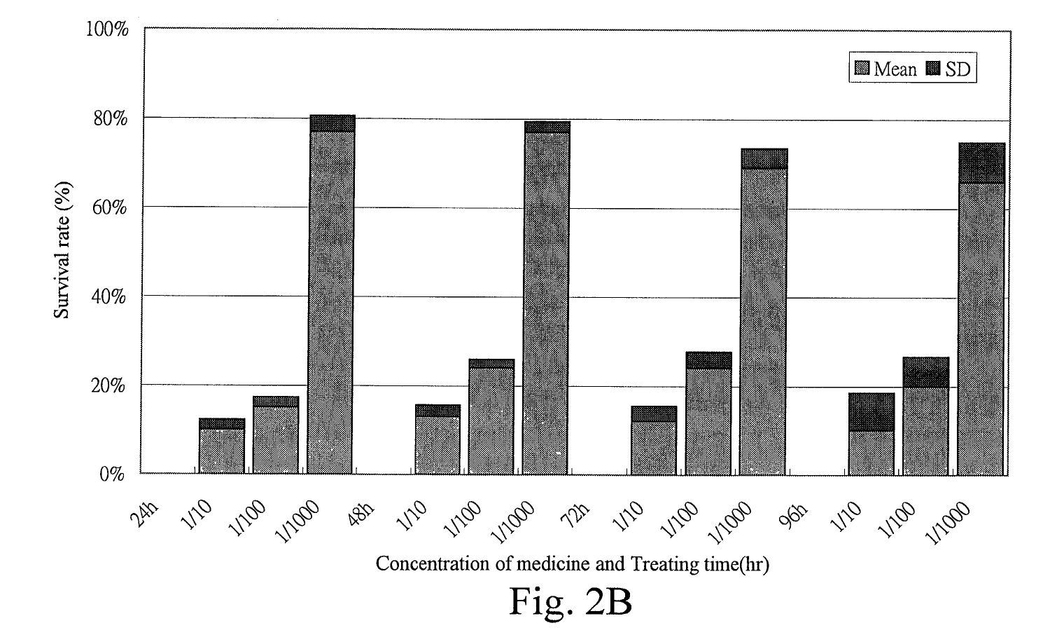

FIGS. 2A and 2B are bar charts illustrating the survival rate of an cervical cancer cell strain, CC-135, treated by cis-Platine or the Chinese herb medicine composition for 24-96 hours according to the present invention. The final concentrations of the Chinese herb medicine composition are 1/10,1/100 and 1/1000 of the extract, respectively. For cis-Platine, the concentrations are 10 times (10 p), 1.0 times (1.0 p) and 0.1 times (0.1 p) of the highest concentration in blood, respectively, i.e. 20, 2.0 and 0.2 mg/ml. As shown in FIG. 2A, when the cervical cancer cell strain CC-135 is treated by 10 p, 1.0 p or 0.1 p anti-cancer drug cis-Platine for 24 hours, the survival rate of CC-135 is about 50%. On the contrary, when the cervical cancer cell strain CC-135 is treated by 1/10 and 1/100 Chinese herb medicine composition extract for 24 hours, the survival rates of CC-135 decrease to 10% and 20%, respectively, as shown on FIG. 2B. Therefore, the results indicate that the inhibition effect of the Chinese herb medicine composition on the cervical cancer cell is much stronger than that of the anti-cancer drug cis-Platine.

Embodiment 3

In this embodiment, the methods for cell culture, sub-culture, experimental treatments, cell activity determination and calculation of the cell survival rate are the same as those of the Embodiment 1 except the cell strain is an ovary cancer cell strain OC-117.

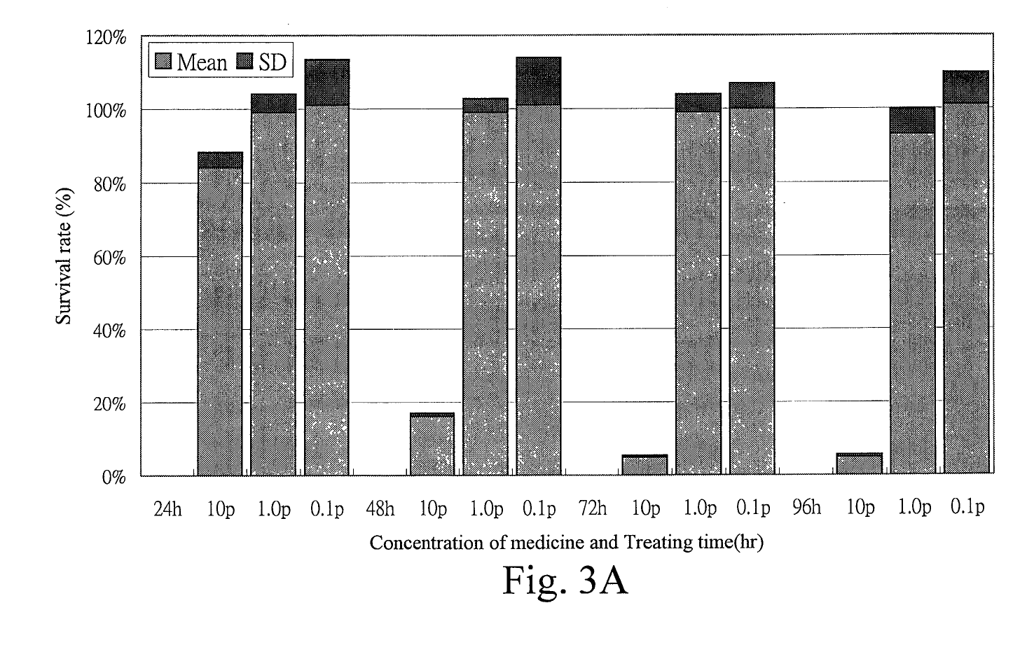

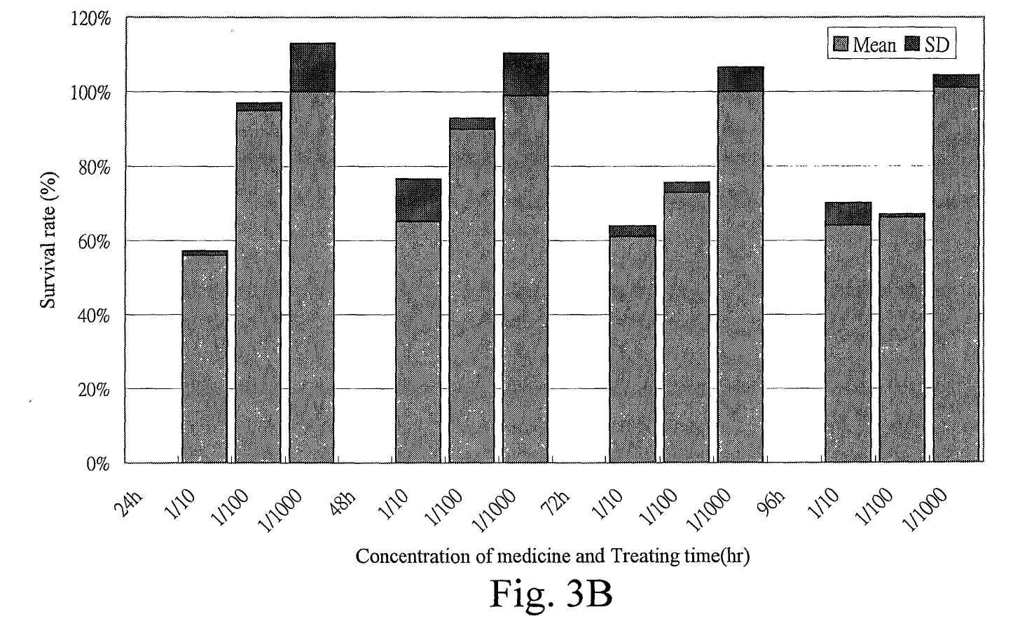

FIGS. 3A and 3B are bar charts illustrating the survival rate of an ovary cancer cell strain, OC-117, treated by cis-Platine or the Chinese herb medicine composition for 24-96 hours according to the present invention. The final concentrations of the Chinese herb medicine composition are 1/10,1/100 and 1/1000 of the extract, respectively. For cis-Platine, the concentrations are 10 times (10 p), 1.0 times (1.0 p) and 0.1 times (0.1 p) of the highest concentration in blood, respectively, i.e. 20, 2.0 and 0.2 mg/ml. As shown in FIG. 3A, only the ovary cancer cell strain OC-117 is treated by 10 p anti-cancer drug cis-Platine for over 48 hours, the inhibition effect of cis-Platine is significant and the survival rate of OC-117 is below 20%. On the contrary, when the ovary cancer cell strain OC-117 is treated by 1/10 and 1/100 Chinese herb medicine composition extract for 24 and 96 hours respectively, the survival rates of OC-117 decrease to about 60% as shown in FIG. 3B. Therefore, the results indicate that the Chinese herb medicine composition has significant inhibition effect on the ovary cancer cell.

### Embodiment 4

In this embodiment, the methods for cell culture, sub-culture, experimental treatments, cell activity determination and calculation of the cell survival rate are the same as those of the Embodiment 1 except the cell strain is a bladder cancer cell strain Bladder-3.

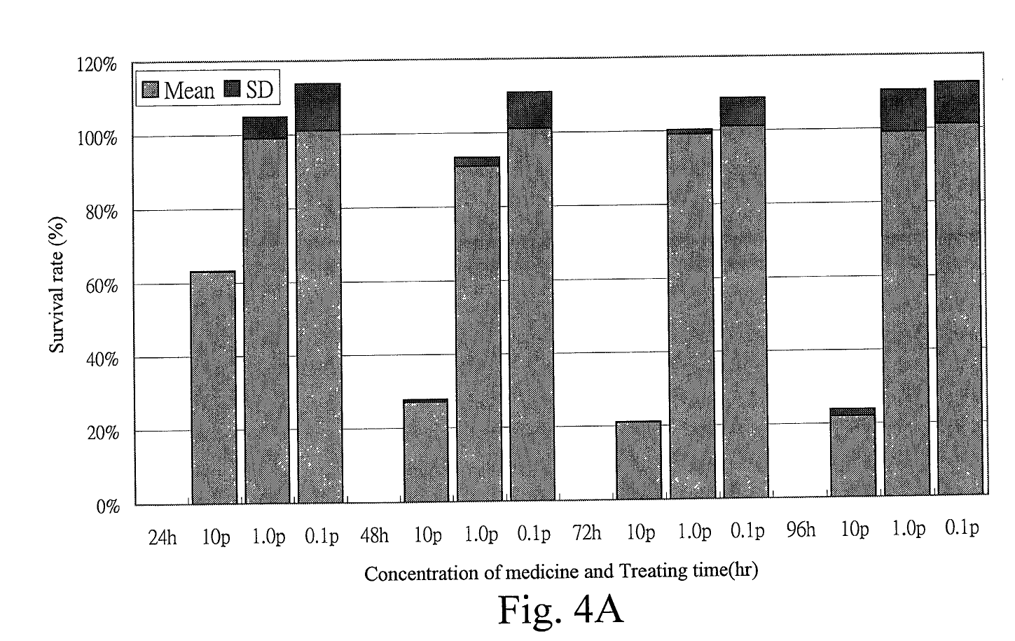

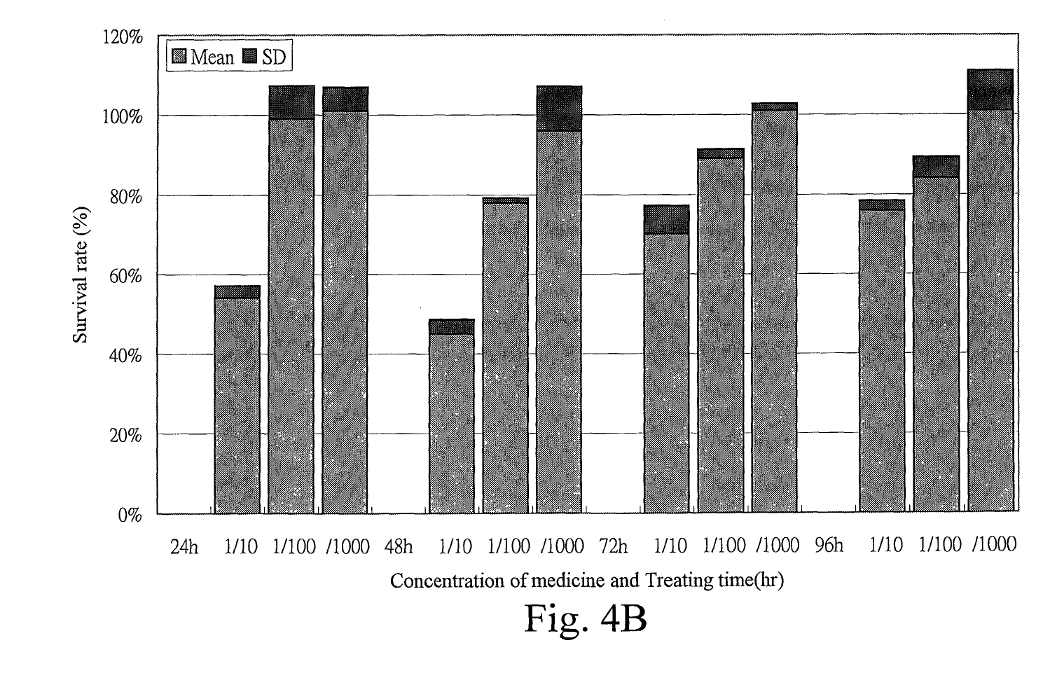

FIGS. 4A and 4B are bar charts illustrating the survival rate of an bladder cancer cell strain, Bladder-3, treated by cis-Platine or the Chinese herb medicine composition for 24-96 hours according to the present invention. The final concentrations of the Chinese herb medicine composition are 1/10,1/100 and 1/1000 of the extract, respectively. For cis-Platine, the concentrations are 10 times (10 p), 1.0 times (1.0 p) and 0.1 times (0.1 p) of the highest concentration in blood, respectively, i.e. 20, 2.0 and 0.2 mg/ml. As shown in FIG. 4A, only the bladder cancer cell strain Bladder-3 is treated by 10 p anti-cancer drug cis-Platine for over 48 hours, the result shows the significant inhibition effect of cis-Platine on Bladder-3. The Chinese herb medicine composition has better inhibition effect on the bladder cancer cell (the survival rate is about 50%) when the bladder cancer cell strain Bladder is treated by 1/10 Chinese herb medicine composition extract for 48 hours as shown in FIG. 4B. In addition, when the bladder cancer cell strain Bladder-3 is treated by 1/100 Chinese herb medicine composition extract for 48 hours, the survival rate of Bladder-3 decreases to 80%. Therefore, the Chinese herb medicine composition has significant inhibition effect on the bladder cancer cell.

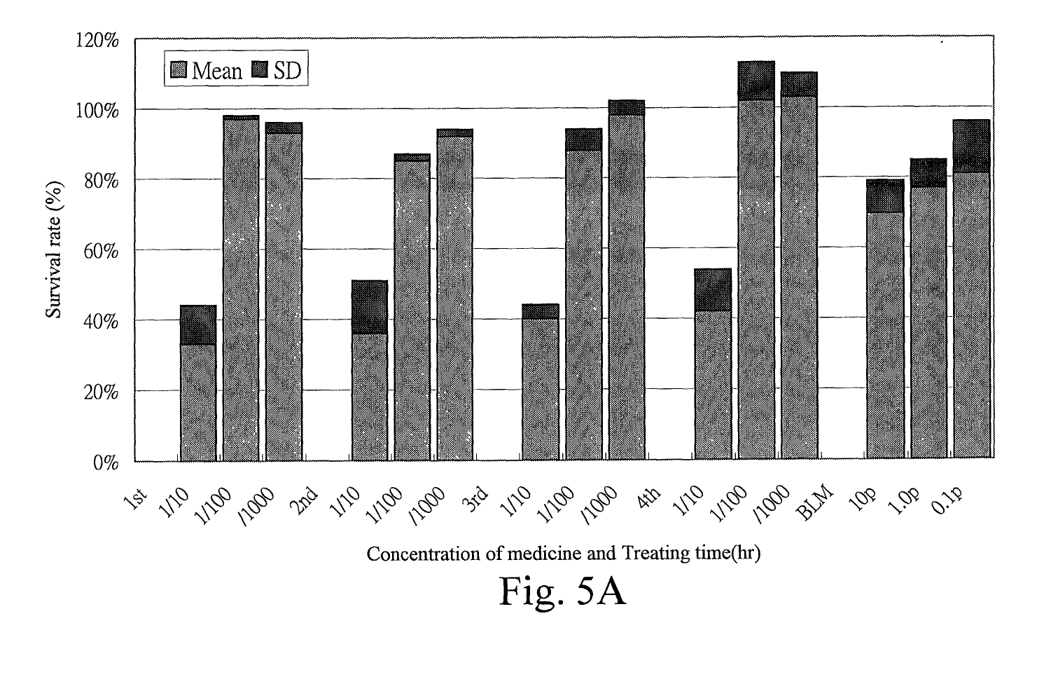

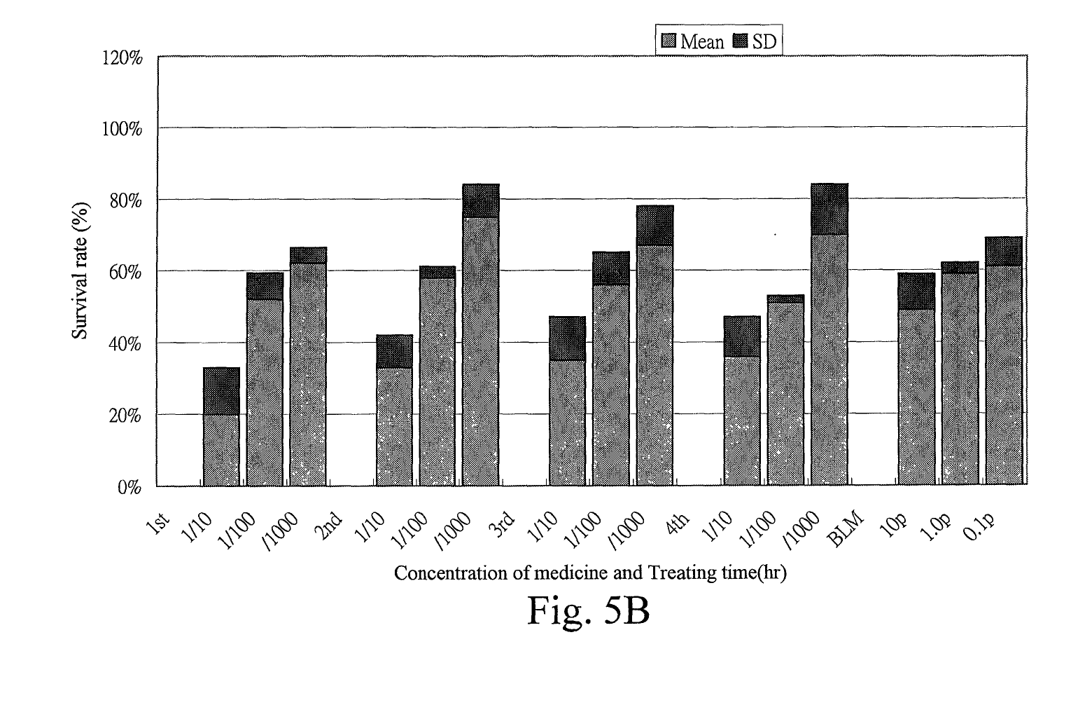

FIGS. 5A and 5B are bar charts illustrating the survival rate of a cervical cancer cell strain, CC-7T, treated for 6 and 8 hours respectively by an extracted liquid of Chinese herb medicine composition stored for different days according to the present invention. The Chinese herb medicine composition extracts prepared from previous procedure, are diluted with 0.9% NaCl to 1/10, 1/100 and 1/1000, and then stored at 4° C. for 1, 2, 3 and 4 weeks, respectively, and then used to treated the cervical cancer cell strain CC-7T for 6 or 8 hours. The anti-cancer drug Bleomycin (BLM) is used as a control test of the Chinese herb medicine composition. The concentrations of Bleomycin are 10 times (10 p), 1.0 time (1.0 p), and 0.1 times (0.1 p) of the highest concentration in blood, i.e. 30, 3.0, and 0.3 mg/ml.

As shown in FIGS. 5A and 5B, one to four week storages do not affect the inhibition effect of Chinese herb medicine composition on the cervical cancer cell strain CC-7T. Furthermore, when the cervical cancer cell strain CC-7T is treated by 1/10 and 1/100 Chinese herb medicine composition extract for 8 hours, the survival rates of CC-7T decrease to 40% and 60%, respectively.

Accordingly, the Chinese herb medicine composition of the present invention can effectively inhibit the cancer cells and be applied in the cancer prophylaxis and therapy. In addition, the present invention also has the following advantages:

1. Because each component of the Chinese herb medicine composition according to the present invention is customarily used Chinese herb, it is no doubt that the composition is safe to be taken.

2. Each component of the Chinese herb medicine composition according to the present invention has been used to cure many malignant furuncles and local inflamed, fever and pain without any ill side-effect for a long time. Therefore, when the Chinese herb medicine composition according to the present invention applied in the cancer prophylaxis and therapy, the ill side-effects resulted from the clinical anti-cancer drugs can be avoided.

3. The Chinese herb medicine composition according to the present invention can be used in the cervical cancer prophylaxis and therapy by external using, such as rinsing and spraying. Therefore, both safety and convenience are considered.

4. The Chinese herb medicine composition according to the present invention can be used by the combination of orally taking and external using, so the inhibition effect on the cancer cell will increase.

5. The Chinese herb medicine composition according to the present invention can be prepared to be an extract or further be a dry powder, so it is easy to use and store for users.

In sum, the Chinese herb medicine composition applied in the cancer prophylaxis and therapy according to the present invention has safety, convenience, and effectiveness without ill side-effects.

While the invention has been described in terms of what are presently considered to be the most practical and preferred embodiments, it is to be understood that the invention need not to be limited to the disclosed embodiment. On the contrary, it is intended to cover various modifications and similar arrangements included within the spirit and scope of the appended claims which are to be accorded with the broadest interpretation so as to encompass all such modifications and similar structures.

What is claimed is:

1. A Chinese herb medicine composition for a cancer prophylaxis and therapy comprising Honeysuckle Flower, Mother Chrysanthemum Flower, Dandelion Herb, Viola Yedoensis and Semiaquilegia adosoides (DC.) makino.

2. The composition according to claim 1, wherein said Chinese herb medicine composition is composed with a special ratio, and said special ratio of Honeysuckle Flower:Mother Chrysanthemum Flower:Dandelion Herb Viola Yedoensis: Semiaquilegia adosoides (DC.) makino by weight is 1:1:1:1:1.

3. The composition according to claim 1, wherein said cancer is selected from a group consisting of cervical cancer, esophagus cancer, ovary cancer and bladder cancer.

4. The composition according to claim 1, wherein said Chinese herb medicine composition is one of an extracted liquid and a dry powder.

5. The composition according to claim 4, wherein said dry powder is formed from said extracted liquid by freeze drying.

6. The composition according to claim 4, wherein said extracted liquid is one of a direct extracted liquid and an extracted concentrate.

7. The composition according to claim 6, wherein said extracted liquid is used directly.

8. The composition according to claim 6, wherein said extracted liquid is used after diluting said direct extracted liquid.

9. The composition according to claim 1, wherein said Chinese herb medicine composition is used by one selected from a group consisting of orally taking, external using and the combination thereof.

10. A Chinese herb medicine composition for a cancer prophylaxis and therapy comprising Dandelion Herb.

11. The composition according to claim 10 further comprising Honeysuckle Flower, Mother Chrysanthemum Flower, Viola Yedoensis and Semiaquilegia adosoides (DC.) makino.

12. The composition according to claim 11, wherein said Chinese herb medicine composition is composed with a special ratio, and said special ratio of Honeysuckle Flower:Mother Chrysanthemum Flower: Dandelion Herb: Viola Yedoensis: Semiaquilegia adosoides (DC.) makino by weight is 1:1:1:1:1.
